(19) United States
(12) Patent Application Publication
(10) Pub. No.: US 2014/0276570 A1
(43) Pub. Date: Sep. 18, 2014

(54) GRADUAL TRANSITION BETWEEN BASAL RATES IN AN AMBULATORY DRUG DELIVERY DEVICE

(71) Applicant: TANDEM DIABETES CARE, INC., San Diego, CA (US)

(72) Inventor: SEAN SAINT, San Diego, CA (US)

(21) Appl. No.: 13/837,777

(22) Filed: Mar. 15, 2013

(51) Int. Cl. A61M 5/172

(52) U.S. Cl. CPC A61M 5/1723
USPC 604/506; 604/151

(57) ABSTRACT

A controller in an ambulatory drug delivery device automatically creates gradual transitions between basal rates entered by a user. The user may enter at least two target basal rates and two corresponding durations for delivering a medication at each rate. Systems and methods for such drug delivery are described.

### BACKGROUND

One way in which current infusion pumps deliver medication is according to basal rate profiles in which the basal rates change in steps over time, with sharp, almost instantaneous transitions between different basal rates. The human body usually does not abruptly change physiological needs dramatically minute to minute, and as such, instantaneous transitions between different basal rates do not reflect the needs of a patient's body.

In addition, patients tend to program only a relatively small number of varying basal rates into a profile of an insulin pump, at the most. This decision to use a basal rate profile that has fewer changes of basal rate during a day may be due to a reluctance to spend too much time perfecting a basal rate profile when an approximation improves the patient's condition enough to satisfy the patient and the patient's physician and/or caregiver. However, programming more basal rates into a profile may decrease the difference between the basal rate profile and the patient's actual needs. Therefore, it would be desirable to provide a system that more easily allows users to mimic the actual basal rate profile that their bodies need.

### SUMMARY

Provided herein are systems for administering medication to a patient that may include an infusion pump and software associated with the infusion pump that is configured to automatically provide a basal medication dosage profile that is based upon input from a user. The basal medication dosage profile may include at least two target basal rates, a first duration for administering a medication to the patient at a first target basal rate, and a second duration for administering the medication at a second target basal rate. The basal medication dosage profile provided by the software may include at least one transition period to gradually transition between the target basal rates, in which the at least one transition period is independent of the target rates.

In some embodiments, the transition period may be set by the software. Alternatively, the transition period may be set by the user. The infusion pump can be configured to administer the medication such that a first volume administered in a first does is either the same as or is different from a second volume administered in a second dose. In some embodiments of the system, a slope of a plot of the basal medication dosage profile provided by the software may be non-zero and not infinity at the at least one transition period. The at least one transition period may occur over a fixed time period. In some embodiments of the system, the basal medication dosage profile may include at least two transition periods, in which each transition period has a duration that is constant within the basal medication dosage profile. The infusion pump may be configured to administer medication doses at fixed time intervals. In such embodiments, the duration of each transition period may be an amount of time (T) that yields a whole number when divided by a duration (t) of the fixed dosage time intervals. Furthermore, in some such embodiments, the duration (t) of the fixed dosage time intervals is 5 minutes. The duration of each transition period (T) may be, for example, 15 minutes, 30 minutes, 60 minutes, 90 minutes, or 120 minutes. In some embodiments, the duration of each transition period (T) may be 60 minutes. In some embodiments of the system, the user may provide input using a user interface located on the infusion pump or on a remote device.

In some embodiments, a method of delivering a medicament to a patient is provided. The method may include accepting, from a user via software associated with an infusion pump, at least two rates of medicament delivery and periods of time during a 24 hour period corresponding to each rate of medicament delivery. The method may also include generating a rate profile based upon the at least two rates of medicament delivery and periods of time using the software associated with the infusion pump and providing the rate profile to a processor associated with the infusion pump for direct administration of the medicament to the patient. In the method, the rate profile may include a transition period between the at least two rates of medicament delivery, the transition period being independent of the at least two rates of medicament delivery provided by the user.

The transition period can have a duration chosen by the user or the software. The infusion pump may be configured to administer medicament doses at fixed time intervals. In some such embodiments, the duration of each transition period may be an amount of time (T) that yields a whole number when divided by a duration (t) of the fixed dosage time intervals. Further, the duration (t) of the fixed dosage time intervals may be 5 minutes. In some embodiments of the method, the pump may administer the medication in a different volume for each dose administered during transition periods. The method may further include refining the basal rate profile. In such embodiments, further refining the basal rate profile may include the user providing additional information. The additional information may include more basal rate intervals, new values for existing basal rate intervals, or both.

Other features and advantages should be apparent from the following description of various embodiments, which illustrate, by way of example, the principles of the disclosure.

### BRIEF DESCRIPTION OF THE DRAWINGS

FIG. 1A is a schematic of an exemplary basal rate profile that would be used in current prior art systems;

FIG. 1B is a schematic of exemplary basal rate profiles illustrating gradual transitions between basal rates;

FIG. 2 is a schematic of a second type of exemplary basal rate profiles; and

FIG. 3 is a flow chart of an exemplary method for automatically providing a basal rate profile with gradual transitions between basal rates in a drug delivery device.

### DETAILED DESCRIPTION

Described herein are methods of delivering a medication to a patient that accurately address the changing needs of the patient's body over the course of day, specifically the ambulatory delivery of insulin to a diabetic patient. A feature programmed in a medication infusion pump, such as an insulin pump, allows for gradual transitions from one basal medication dosage rate to another basal medication dosage rate to accommodate the patient's physiologic needs. The resulting basal rate profile can resemble a series of plateaus in dosing rates with line segments of a finite slope connecting the plateaus over a fixed time period for the transition, as opposed to a series of step functions. Systems and apparatus for implementing such methods are also described.

FIGS. 1A and 1B are plots depicting exemplary basal rate profiles for a patient who has five basal rate changes per day. The plot depicts changes in time (minutes) along the x-axis and changes in insulin basal rate, as units per hour (UI/hour), along the y-axis. In these exemplary profiles, the smallest increment or amount of time between changes in basal rates is five minutes. That is to say, the infusion pump in this example administers doses of insulin every five minutes.

FIG. 1A shows a patient's basal rate profile as used by insulin pump systems of the prior art. In FIG. 1A, the basal rate profile is seen to jump dramatically and instantaneously with each basal rate change. For example, in the first transition 110, the basal rate changes by a factor of more than 2, or an increase of more than 100%, in just 5 minutes (300 seconds). In the FIG. 1A conventional basal rate profile of the prior art, the subsequent transitions 120, 130, 140, and 150 each occur in a similarly short amount of time. As mentioned above, these types of abrupt changes do not approximate or mimic physiologic changes in the patient to a satisfactory level.

An embodiment of basal rate profiles that gradually transition between consecutive basal rates according to an embodiment of the present invention is shown in FIG. 1B, laid over the exemplary basal rate profile of FIG. 1A of the prior art. One plot shows a basal rate profile in which transitions between basal rates occur over a period of an hour. Another plot illustrates a basal rate profile in which the transitions occur over a period of two hours. These longer transition periods mean the time during which a target basal rate is administered shortens. For example, in the first elevated basal rate 160 of FIG. 1B, the exemplary basal rate profile with 1-hour transitions allows the insulin pump to dispense insulin at that first elevated basal rate for two hours before beginning the transition to the next rate, and the exemplary basal rate profile with 2-hour transitions allows for dispensing insulin at that first elevated basal rate for only 1 hour.

This shortening of a basal rate period is more dramatic in a shorter basal rate period, such as shown in FIG. 2 for the second elevated basal rate 170. In a conventional basal rate profile (the “current systems” profile in the plot of FIG. 1B), the pump dispenses insulin at the second elevated basal rate 170 for a period of 2 hours. By contrast, the basal rate profile embodiment with 1-hour transitions allows for only 1 hour of dispensing at this second elevated basal rate 170, while the basal rate profile embodiment with 2-hour transitions allows for only one dose at that level. However, the total amount of insulin delivered to the patient over the 24 hour period shown in FIG. 2 (1440 seconds), is the same for the three basal rate profiles shown. This can be confirmed by comparing the area under the curve 180 for each profile.

According to one embodiment, the exemplary basal rate profiles shown in FIG. 1B with transitions of 1 hour or 2 hours are automatically generated in an exemplary insulin pump. A user, such as the patient or the patient's physician, nurse, or other caregiver, may employ a user interface on the insulin pump to designate at least two periods in a 24 hour period with different basal rates to create a basal rate profile for the patient. Software associated with the insulin pump applies transition periods to the basal rate profile to create a modified basal rate profile. The transition periods are greater than the time period between subsequent doses. The transition periods may be predetermined, such as 1 hour or 2 hours. The user may select the duration of the transition periods from a list of available transition periods, or the user may select any time period that is an interval of the pump's pre-set interval between subsequent doses.

Once the software associated with the pump has received the basal rate profile from the user and a duration for the transition periods has been selected, the software generates the modified basal rate profile. The modified basal rate profile indicates to the insulin pump the amount of insulin to be administered at each time point. Each time point in the modified basal rate corresponds to advancing time in equal, predetermined increments, such that there are whole number amount of increments in a 24 hour period. The equal, predetermined increments may be an amount of time that cannot be changed by the user, or alternatively, the user can select the increments.

To determine the basal rate at each time point, the following formula may be applied.

Dc=Dp+((R2−R1)/(T/t))

in which: Dc=current dose rate; Dp=previous dose rate; R1=previous basal rate; R2=next basal rate; T=transition time period; and t=time for each equal, predetermine time increment

For example, in FIG. 1B, to calculate the basal rate at each dose (i.e. Dc for each dose) in the first transition period 110, it is given that the previous basal rate (R1) is 0.6 IU/hour in the first period of time 190. The next basal rate (R2) is 1.25 IU/hour in the second time period 160. As mentioned before, the time for each increment (t) in FIG. 1 is 5 minutes. For the exemplary basal rate profile embodiment with 1 hour transition periods. T=60 minutes. To calculate the first transition dose rate, the difference between the basal rates is divided by the number of intervals over which the transition will take place to arrive at an incremental change value. The incremental change value is then added to the previous dose rate (Dp) to arrive at the current dose rate (Dc). In this instance, R2−R1=0.65 IU/hour, T/t=12, and Dp=0.6 IU/hour. This yields a Dc value of approximately 0.654 IU/hour. This Dc indicates that the pump administers approximately 0.0545 IU in the first transition dose in the first transition period 110 (i.e. Dc multiplied by (1/(60/t)) yields the first transition dose).

For the exemplary basal rate profile embodiment with 2 hour transition periods shown in FIG. 1, T=120, and T/t=24. This change yields a Dc of 0.672 IU/hour and 0.0523 IU to be administered in the first transition dose in the first transition period 110. The more gradual the slope of the basal rate profile plot at the transition points for a given time period, the smaller the incremental change value. The transition time period is independent of the previous basal rate (R1) and the next basal rate (R2), but the combination of the time periods and the values for R1 and R2 will determine the rate of change in the basal rate during the transition period.

The slope of basal rate profiles according to the current disclosure during transition periods can be non-zero and not infinite. The slope during each transition period in basal rate profiles according to the current disclosure can be a constant, finite value, or the value of the slope may vary over the transition period. In contrast, in the basal rate profiles of insulin pump systems of the prior art, the slope of the basal rate profiles during their transition periods is essentially infinite, such that those portions of each basal rate profile are a vertically jumping directly from a current basal rate to the next basal rate with no transition in between.

FIG. 2 shows an embodiment of a second set of exemplary basal rate profiles that correspond more closely to the daily fluctuations of insulin requirements in a patient's body compared to basal rate profiles used with current systems. The basal rate profiles shown in FIG. 2 attempt to mimic a circadian rhythm. In the prior art basal rate profile shown in FIG. 2, there are 20 changes in basal rate. Overlaid on that profile, is one in which the time period for transitions is a half hour (i.e. T=30 minutes, T/t=6) and another basal rate profile in which transitions take place over an hour (i.e. T=60, T/t=12). The exemplary basal rate profile corresponding to current systems shown in FIG. 2 exhibits vertical slopes in the transition periods, as in the exemplary basal rate profile shown in FIG. 1A, and is similarly not a satisfactory approximation of physiological changes. The overlaid exemplary profiles in FIG. 2, those with transition periods of half an hour and an hour, improve on the exemplary basal rate profile that attempts to mimic a circadian rhythm by providing gradual transitions between basal rates.

The formula of the present invention as applied in FIG. 1B to the conventional basal rate profile of FIG. 1A was used to generate the basal rates profile embodiments for the half hour and one hour transition periods in FIG. 2. The numerous basal rate changes that result allow each distinct basal rate period to last no more than 4 hours; most for only one hour. Because of this, the basal rate profile in which the transition period is an hour in this example resembles a polynomial function as opposed to a series of step functions.

To generate the inventive modified basal rate embodiments with transition periods of half an hour or an hour shown in FIG. 2, for example, an insulin pump may accept a series of basal rates and period settings from a user. Alternatively, software associated with the insulin pump may be pre-programmed to generate such basal rate profiles based upon, e.g., known blood glucose variation according to circadian rhythms as categorized by age and/or as user provided patient information. The patient information provided by the user may include, but is not limited to the patient's age, sex, weight, and total daily insulin dose. The circadian rhythm data may be based upon studies in which subjects were separated into groups based upon age. In some instances, the circadian rhythm data may be based upon studies in which more patient characteristics were correlated to the circadian rhythms. As mentioned above, the user may provide a basal rate profile via a user interface, such as a graphical user interface. The patient information provided by the user may also be provided through a user interface.

It should be noted that the insulin pump is capable of, and configured to, dispense any volume needed to create the gradual transitions between basal rates. In some instances, this capability is due to the ability of the insulin pump to dispense insulin in varying amounts with each dose. In other instances, this capability is achieved by administering more than one dose at each time period, including fractional doses. Examples of infusion pumps that can be used in the present invention and various features that can be associated with such pumps include those disclosed in U.S. patent application Ser. No. 13/557,163, U.S. patent application Ser. No. 12/714,299, U.S. patent application Ser. No. 12/538,018, U.S. Provisional Patent Application No. 61/655,883, U.S. Provisional Patent Application No. 61/656,967 and U.S. Pat. No. 8,287,495, each of which is incorporated herein by reference. As mentioned previously, the transition time period is independent of the basal rates adjacent to the transition periods. To accommodate this, the rate of change of the basal rate during each transition period is variable. As such, the insulin pump must be capable of performing as described above.

A user, such as a patient or caregiver, may wish to further fine-tune a patient's basal rate profile in response to the patient's condition. The user may wish to add more basal rate intervals, such that there are more changes in basal rate in a given basal rate profile. Changes to the actual basal rate values may also be something the user may wish to do. Such line-tuning may be done using a user interface. The user interface may be the same user interface used to provide initial information to the insulin pump. The user interface may be located on the insulin pump or on a remote device, such as a remote control (including a dedicated remote control or a mobile device such as a Smartphone), a glucose meter, or another insulin pump and the like.

The user interface may also be an external computing device that only has intermittent communication with the insulin pump, such as a laptop or desktop computer that uses a wireless or hard wired connection to exchange data with the insulin pump. Other types of external computing devices include tablet computers, hand-held computing devices, cellular phones, and the like. Wireless connections can utilize any suitable wireless system, such as Bluetooth, Wi-Fi, radio frequency, Zigbee communication protocols, infrared or cellular phone systems, and can also employ coding or authentication to verify the origin of the information received by either or both the sensor systems and the central monitoring point. Wired connections can include telephone line connections, RS22 connection, USB connection, Firewire® connections, proprietary connections, or any other suitable type of hard-wired connection.

The software associated with the insulin pump that generates basal rate profiles based upon the user provided information may be located on memory and utilize processors on the insulin pump or an external computing device. In some instances, the user provided information is used to generate a basal rate profile on an external device, and that generated basal rate profile is provided to the insulin pump for direct insulin administration.

FIG. 3 is a flow chart of an exemplary method for automatically providing a basal rate profile with gradual transitions between basal rates in a drug delivery device, specifically an insulin pump. In 310, a user inputs two or more basal rates that take effect during different time periods during a day into an insulin pump. The insulin pump controller then automatically calculates the basal rates during transition periods, as well as the associated basal rate profile, as shown in 320. Following this, as shown in 330, the insulin pump dispenses insulin according to the basal rate profile. At this point, the user can observe the condition of a patient as he or she responds to receiving insulin according to the generated basal rate profile. If the user wishes to further refine the basal rate profile, he or she may do so by, for example, providing additional information, as in 340. As mentioned herein above, the transition periods may be chosen by the user or predetermined by the software associated with the insulin pump.

Though the methods and systems herein have been described with respect to a diabetic patient being treated with insulin using an ambulatory insulin pump, it should be appreciated that the methods for providing medicament dosing profiles that mimic a patient's natural rhythms and physiological needs may be applied to other diseases, medicaments (such as, e.g., glucagon and pramlintide), and types of drug-providing apparatus.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, methods of use, embodiments, and combinations thereof are also possible. Therefore the spirit and scope of the appended claims should not be limited to the description of the embodiments contained herein.

1. A system for administering medication to a patient, comprising:
an infusion pump; and
software associated with the infusion pump configured to automatically provide a basal medication dosage profile that is based upon input from a user, the basal medication dosage profile comprising
at least two target basal rates;
a first duration for administering a medication to the patient at a first target basal rate; and
a second duration for administering the medication at a second target basal rate; and
at least one transition period over which a delivered basal rate gradually transitions between the target basal rates.

2. The system of claim 1, wherein the transition period is automatically set by the software.

3. The system of claim 1, wherein the transition period is set by the user.

4. The system of claim 1, wherein the infusion pump is configured to administer the medication such that a first volume administered in a first dose is either the same as or is different from a second volume administered in a second dose.

5. The system of claim 1, wherein a slope of a plot of the basal medication dosage profile as basal rate versus time provided by the software is non-zero and not infinity over the at least one transition period.

6. The system of claim 1, wherein the at least one transition period occurs over a fixed time period.

7. The system of claim 1, wherein the basal medication dosage profile includes at least two transition periods and each transition period has a duration that is constant within the basal medication dosage profile.

8. The system of claim 7, wherein the infusion pump is configured to administer medication doses at fixed time intervals.

9. The system of claim 8, wherein the duration of each transition period is an amount of time (T) that yields a whole number when divided by a duration (t) of the fixed dosage time intervals.

10. The system of claim 9, wherein the duration (t) of the fixed dosage time intervals is 5 minutes.

11. The system of claim 10, wherein the duration of each transition period (T) is 15 minutes, 30 minutes, 60 minutes, 90 minutes, or 120 minutes.

12. The system of claim 10, wherein the duration of each transition period (T) is 60 minutes.

13. The system of claim 1, wherein the user provides input using a user interface located on the infusion pump or on a remote device.

14. A method of delivering a medicament to a patient, comprising:
accepting, from a user via software associated with an infusion pump, at least two rates of medicament delivery and periods of time during a 24 hour period corresponding to each rate of medicament delivery;
generating a rate profile based upon the at least two rates of medicament delivery and periods of time using the software associated with the infusion pump; and
providing the rate profile to a controller associated with the infusion pump to direct administration of the medicament to the patient,
wherein the rate profile comprises a transition period between the at least two rates of medicament delivery over which a delivered rate gradually transitions between the two rates.

15. The method of claim 14, wherein the transition period has a duration chosen by the user.

16. The method of claim 14, wherein the transition period has a duration automatically determined by the software.

17. The method of claim 14, wherein the infusion pump is configured to administer medicament doses at fixed time intervals.

18. The method of claim 17, wherein the duration of each transition period is an amount of time (T) that yields a whole number when divided by a duration (t) of the fixed dosage time intervals.

19. The system of claim 18, wherein the duration (t) of the fixed dosage time intervals is 5 minutes.

20. The system of claim 14, wherein the pump administers the medication in a different volume for each dose administered during transition periods.

21. The method of claim 14, further comprising further refining the basal rate profile.

22. The method of claim 21, wherein further refining the basal rate profile comprises the user providing additional information.

23. The method of claim 22, wherein the additional information comprises more basal rate intervals, new values for existing basal rate intervals, or both.
